(19) United States
(12) Patent Application Publication
(10) Pub. No.: US 2011/0071130 A1
(43) Pub. Date: Mar. 24, 2011

(54) 2-AMINOBENZIMIDAZOLES FOR TREATING NEURODEGENERATIVE DISEASES

(75) Inventors: Andrew G. Cole, Robinsville, NJ (US); Brian F. McGuinness, Plainsboro, NJ (US); Guizhen Dong, Dayton, NJ (US); Ian Henderson, Hopewell, NJ (US)

(73) Assignee: Pharmacopeia, Inc., Princeton, NJ (US)

(21) Appl. No.: 12/299,515

(22) PCT Filed: May 7, 2007
(86) PCT No.: PCT/US07/68318

§ 371 Date: Dec. 8, 2010

Related U.S. Application Data
(60) Provisional application No. 60/746,675, filed on May 8, 2006.

(51) Int. Cl. A61K 31/4184; C07D 235/30; C07D 401/06; A61K 31/454; C07D 403/06; C07D 413/06; A61K 31/5377; C07D 405/12; A61P 25/00; A61P 25/02; A61P 25/28; A61P 9/00; A61P 9/10; A61P 25/04; A61P 25/20; A61P 27/02; A61P 25/16; A61P 25/14

(52) U.S. Cl. 514/210.21; 548/307.4; 514/395; 546/199; 514/322; 548/306.1; 544/139; 514/234.5; 548/304.7

(57) ABSTRACT

The invention relates to 2-aminobenzimidazoles useful in treating disorders that are mediated by A₂ₐ receptor function, including neurodegenerative diseases including Parkinson's disease and inflammation. The compounds have general formula I:

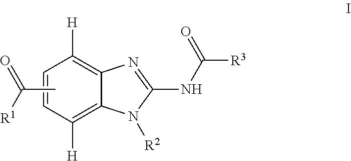

### FIELD OF THE INVENTION

The invention relates to substituted 2-aminobenzimidazoles useful in treating disorders that are mediated by adenosine receptor function, including neurodegenerative diseases and inflammation.

### BACKGROUND OF THE INVENTION

Adenosine is a modulator of multiple physiological functions, including cardiovascular, neurological, respiratory and renal functions. Adenosine mediates its effects through specific G-protein coupled receptors A₁, A₂ₐ, A_{2b} and A₃.

Adenosine 2a (A₂ₐ) receptor antagonists useful in the treatment of Parkinson's disease have been disclosed in U.S. Pat. No. 6,875,772 and U.S. Pat. No. 6,787,541. These disclosures are incorporated herein by reference as they relate to utility.

### SUMMARY OF THE INVENTION

In one aspect the present invention provides compounds according to formula I, useful as adenosine 2a (A2a) receptor antagonists:

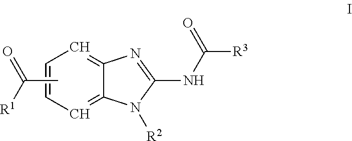

In these compounds
R¹ is selected from the group consisting of OR⁴, N(R⁵)(CH₂)ₙR⁶ and N(R⁵)R⁷;
R² is selected from the group consisting of C₃-C₂₀ hydrocarbon in which from one to three —CH₂— are replaced by —O—;
R³ is selected from the group consisting of aryl, arylalkyl, heteroaryl, heteroarylalkyl, R⁹-substituted aryl, R⁹-substituted arylalkyl, R⁹-substituted heteroaryl and R⁹-substituted heteroarylalkyl, wherein R⁹ represents from 1 to 3 substituents independently selected from cyano, methyl, methoxy, hydroxy, nitro and halogen;
R⁴ is selected from the group consisting of H, C₁-C₂₀ hydrocarbon, heteroaryl, heteroarylalkyl, substituted alkyl, substituted aryl, substituted arylalkyl, substituted heteroaryl and substituted heteroarylalkyl;
R⁵ is selected from the group consisting of H and C₁-C₄ alkyl;
n is an integer selected from 1-4;
R⁶ is selected from the group consisting of aryl, heteroaryl, substituted aryl and substituted heteroaryl; or
when n is 2, 3 or 4, R⁶ may additionally be alkoxy, aryloxy or substituted aryloxy; and
R⁷ is H or C₁-C₂₀ hydrocarbon,
or R⁵ and R⁷, together with the nitrogen atom to which they are attached, form a 4-7 membered optionally substituted monocyclic ring or an 8-14 membered optionally substituted bicyclic ring, wherein each monocyclic or bicyclic ring optionally contains an additional 1 to 3 heteroatoms chosen from N, O and S. The R⁵—N—R⁷ ring or rings may be aromatic or non-aromatic;
with the provisos that:
(1) when R² is —(CH₂)₃—OCH₃, —CO—R¹ is at the 5-position of benzimidazole ring, and R¹ is —N(CH₃)-cyclohexyl, R³ is not thien-2-yl,
(2) when R² is —(CH₂)₃—OCH₃, —CO—R¹ is at the 7-position of benzimidazole ring, and R¹ is —NH-benzyl, R³ is not 3-cyanophenyl, and
(3) when R² is —(CH₂)₃—OCH₃, —CO—R¹ is at the 5-position of benzimidazole ring, and R¹ is —NH—CH₂-(3-methoxyphenyl), R³ is not 1-cyanocyclopropyl, 3-N,N-dimethylaminophenyl, 3-trifluoromethylphenyl, or 2-methoxyethyl.

In another aspect, the invention relates to pharmaceutical compositions comprising a therapeutically effective amount of at least one compound of general formula I or a pharmaceutically acceptable salt thereof; and a pharmaceutically acceptable carrier.

The compounds and pharmaceutical compositions described herein are useful in methods for preventing and treating a condition for which an antagonist of adenosine 2a receptor is indicated.

In a third aspect, the invention relates to a method for treating a disease by antagonizing a response mediated by adenosine 2a receptors. The method comprises bringing into contact with an adenosine receptor at least one compound of general formula I or a pharmaceutically acceptable salt thereof.

In yet another aspect the present invention relates to a method of treating a disease mediated by an adenosine receptor in a subject in need thereof comprising administering to the subject a therapeutically effective amount of at least one compound of general formula I or a pharmaceutically acceptable salt thereof. Treating a disorder mediated by an adenosine receptor includes treating disorders associated with A₂ₐ receptors and one or more additional adenosine receptors, such as A₁, A_{2b} or A₃ receptors.

The compounds of the present invention are useful in effecting neuroprotection and as such the present invention provides a method of neuroprotection in a subject in need thereof comprising administering to the subject a therapeutically effective amount of at least one compound of general formula I or a pharmaceutically acceptable salt thereof.

Other indications in which the adenosine antagonists are useful include central nervous system disorders, neurodegenerative diseases, cardiovascular disorders, and diabetes.

The compounds of the present invention are useful in combination with one or more of (1) an agent useful in the treatment of Parkinson's disease, (2) an agent useful in the treatment of movement disorders, (3) an agent useful in the treatment of depression.

### DETAILED DESCRIPTION OF THE INVENTION

Throughout this specification the substituents are defined when introduced and retain their definitions.

It has now been found that compounds of general formula I are potent antagonists of the adenosine 2a (A₂ₐ) receptor:

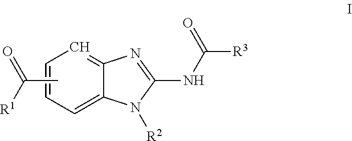

In these compounds R¹ is selected from the group consisting of OR⁴, N(R⁵)(CH₂)ₙR⁶ and N(R⁵)R⁷. The compounds may thus be conveniently divided into two subgenera, Id, in which —C(O)R¹ is attached at the 5- or 6-position of the benzimidazole ring, and Ie, in which —C(O)R¹ is attached at the 7-position of the benzimidazole ring:

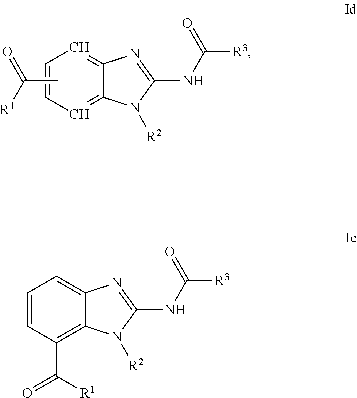

Subgenus Id can further be subdivided into subgenera Ia, the esters, and Ib and II, the amides:

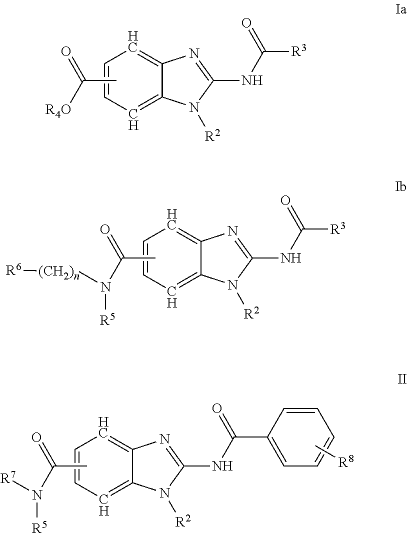

In the subgenus II, R⁷ is H or C₁-C₂₀ hydrocarbon; and R⁸ is halogen, cyano, C₁-C₄ alkoxy, hydroxyl, C₁-C₄ alkyl or trifluoromethyl; or R⁵ and R⁷, together with the nitrogen atom to which they are attached, form a 4-7 membered optionally substituted monocyclic heterocyclic ring or an 8-14 membered optionally substituted bicyclic heterocyclic ring, wherein each monocyclic or bicyclic ring optionally contains an additional 1 to 3 heteroatoms chosen from N, O and S. The R⁵—N—R⁷ ring or rings may be aromatic or non-aromatic.

Throughout the specification and claims, the statement that R² is selected from the group consisting of C₃-C₂₀ hydrocarbon in which from one to three —CH₂— are replaced by —O— means that a —CH₂— unit has been replaced by an oxygen to yield an ether, and does not include replacement of a group ═CH₂ by oxygen to form a carbonyl, nor does it include insertion of an oxygen between a carbon and a hydrogen (or replacement of a CH₂ unit of a methyl moiety) to form a hydroxyl group. Thus, in many embodiments R² is C₃-C₂₀ oxaalkyl and R³ is heteroaryl or substituted phenyl. As will be evident to the artisan, the oxygen of the oxaalkyl will not normally be situated in the positions α and β to the ring.

In certain embodiments R⁵ is H.

In certain embodiments n is 1 and R⁶ is substituted aryl. In others, n is 2 or 3 and R⁶ is selected from the group consisting of aryl, heteroaryl, substituted aryl, substituted heteroaryl, alkoxy, aryloxy and substituted aryloxy.

In another aspect the present invention provides a method of treating a disorder, which is mediated by adenosine 2a (A₂ₐ) receptor function, which comprises administering to a subject in need of such treatment a therapeutically effective amount of a compound of formula I. It also encompasses a method of treating a disorder associated with A₂ₐ receptor and one or more of A₁, A_{2b} or A₃ receptors. All of the compounds falling within the foregoing parent genera and their subgenera are useful as adenosine receptor antagonists.

For convenience and clarity certain terms employed in the specification, examples and claims are described herein.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

C₁ to C₂₀ hydrocarbon includes alkyl, cycloalkyl, alkenyl, alkynyl, aryl and combinations thereof. Examples include phenethyl, cyclohexylmethyl, camphoryl, adamantyl and naphthylethyl.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. When used to describe a substituent on an aryl ring, alkoxy also is intended to encompass methylene dioxy.

Alkoxyalkyl refers to ether groups of from 3 to 8 atoms of a straight, branched, cyclic configuration and combinations thereof attached to the parent structure through an alkyl. Examples include methoxymethyl, methoxyethyl, ethoxypropyl, and the like.

Alkoxyaryl refers to alkoxy substituents attached to an aryl, wherein the aryl is attached to the parent structure.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene and naphthalene, and according to the invention benzoxalane and residues in which one or more rings are aromatic, but not all need be. The 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl refers to a substituent in which an aryl residue is attached to the parent structure through alkyl. Examples are benzyl, phenethyl and the like. Heteroarylalkyl refers to a substituent in which a heteroaryl residue is attached to the parent structure through alkyl. Examples include, e.g., pyridinylmethyl, pyrimidinylethyl and the like.

Heterocycle means a cycloalkyl or aryl residue in which from one to three carbons is replaced by a heteroatom selected from the group consisting of N, O and S. The nitrogen and sulfur heteroatoms may optionally be oxidized, and the nitrogen heteroatom may optionally be quaternized. Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. According to convention, the suffix “yl” indicates the moiety in question appearing as a residue on a parent structure. Thus, for example, heterocyclyl means a heterocycle appearing as a substituent rather than a parent. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygens, as well as other heteroatoms. A sulfur heterocycle is a heterocycle containing at least one sulfur in the ring; it may contain additional sulfurs, as well as other heteroatoms. A nitrogen heterocycle is a heterocycle containing at least one nitrogen in the ring; it may contain additional nitrogens, as well as other heteroatoms. Oxygen heteroaryl is a subset of oxygen heterocycle; examples include furan and oxazole. Sulfur heteroaryl is a subset of sulfur heterocycle; examples include thiophene and thiazine. Nitrogen heteroaryl is a subset of nitrogen heterocycle; examples include pyrrole, pyridine and pyrazine.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The terms “halogen” and “halo” refer to fluorine, chlorine, bromine or iodine.

Some of the compounds described herein may contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as, their racemic and optically pure forms. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration; thus a carbon-carbon double bond depicted arbitrarily herein as trans may be Z, E or a mixture of the two in any proportion.

The graphic representations of racemic, ambiscalemic and scalemic or enantiomerically pure compounds used herein are taken from Maehr, J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, chlorine and iodine include ³H, ¹⁴C, ³⁵S, ¹⁸F, ³⁶Cl and ¹²⁵I, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention can generally be prepared by methods well known to those skilled in the art. Conveniently, such radiolabeled compounds can be prepared by carrying out the procedures disclosed in the Examples by substituting a readily available radiolabeled reagent for a non-radiolabeled reagent. Because of the high affinity for the A2a receptor, radiolabeled compounds of the invention are useful for A2a receptor assays.

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups” Suitable groups for that purpose are discussed in standard textbooks in the field of chemistry, such as Protective Groups in Organic Synthesis by T. W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, for example in the case of suitably substituted benzimidazole ring compounds, are either commercially available, synthesized as described in the examples or may be obtained by the methods well known to persons of skill in the art.

The present invention further provides pharmaceutical compositions comprising as active agents, the compounds described herein.

As used herein a “pharmaceutical composition” refers to a preparation of one or more of the compounds described herein, or physiologically acceptable salts or solvents thereof, with other chemical components such as physiologically suitable carriers and excipients.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Compounds that antagonize the adenosine receptor can be formulated as pharmaceutical compositions and administered to a mammalian subject, such as a human patient in a variety of forms adapted to the chosen route of administration, i.e., orally or parenterally, by intravenous, intramuscular, topical, transdermal or subcutaneous routes.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating may be useful as it is may be desirable to prevent exposure of the compounds of the invention to the gastric environment.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds of the invention may be formulated in aqueous solutions, preferably in physiologically compatible buffers such as Hank's or Ringer's solution or physiological saline buffer. For transmucosal and transdermal administration, penetrants appropriate to the barrier to be permeated may be used in the composition. Such penetrants, including for example DMSO or polyethylene glycol, are known in the art.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

The compounds of the present invention may also be formulated in rectal compositions such as suppositories or retention enemas, using, e.g., conventional suppository bases such as cocoa butter or other glycerides.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on many factors including the subject being treated, the severity of the affliction, the manner of administration, the judgment of the prescribing physician. The compounds of the invention may be administered orally or via injection at a dose from 0.001 to 2500 mg/kg per day. The dose range for adult humans is generally from 0.005 mg to 10 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

As used herein, and as would be understood by the person of skill in the art, the recitation of “a compound” is intended to include salts, solvates and inclusion complexes of that compound. The term “solvate” refers to a compound in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Inclusion complexes are described in Remington: Time Science and Practice of Pharmacy 19th Ed. (1995) volume 1, page 176-177, which is incorporated herein by reference. The most commonly employed inclusion complexes are those with cyclodextrins, and all cyclodextrin complexes, natural and synthetic, are specifically encompassed within the claims.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended herein.

It should be understood that in addition to the ingredients particularly mentioned above, the formulations of this invention may include other agents conventional in the art having regard to the type of formulation in question, for example those suitable for oral administration may include flavoring agents.

The compositions may be presented in a packaging device or dispenser, which may contain one or more unit dosage forms containing the active ingredient. Examples of a packaging device include metal or plastic foil, such as a blister pack and a nebulizer for inhalation. The packaging device or dispenser may be accompanied by instructions for administration. Compositions comprising a compound of the present invention formulated in a compatible pharmaceutical carrier may also be placed in an appropriate container and labeled for treatment of an indicated condition.

The compounds and compositions of the present invention may be used as a stand alone treatment or administered in combination with additional agents useful in treating neurodegenerative disorders, movement disorders, depression, for example in combination with L-dopa.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within any number of hours of each other or within any number or days or weeks of each other. In some cases even longer intervals are possible.

While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

As antagonists of A₂ₐ receptors, the compounds of formula I have utility in treating and preventing inter alia neurodegenerative disorders and depression. The compounds and compositions can be used advantageously in combination with other agents useful in treating neurodegenerative disorders and depression. For example, a compound or compounds of formula I may be used in preparing a composition further comprising L-dopa and or caffeine for utility in the treatment of Parkinson's and related diseases.

The compounds of the present invention are useful in inhibiting the activity of adenosine receptors or in inhibiting adenosine receptor-mediated activity and are useful in treating complications arising therefrom.

In some embodiments the compounds of the present invention are useful in inhibiting the activity of A₂ₐ receptors or in inhibiting A₂ₐ receptor-mediated activity and are useful in treating complications arising therefrom.

According to the present invention, the A₂ₐ receptor antagonists may be administered prophylactically, i.e., prior to onset of a neurological disorder, or they may be administered after onset of the disorder, or at both times.

A₂ₐ antagonists have been shown to produce an increase in locomotor activity, a decrease of neuroleptic-induced catalepsy, decrease of MPTP-induced hypomotility, reversal of cocaine withdrawal-induced anhedonia and several indications of neuroprotection in response to brain injury. These observation support therapeutic indications of A₂ₐ antagonists for inter alia Parkinson's disease (PD) and cocaine abuse, and neurodegenerative disorders such as Alzheimer's disease.

A₂ₐ antagonists, such as SCH 58261 and KW-6002, are particularly compelling for the treatment of PD since they not only enhance locomotor activity in animal models as a stand-alone treatment, but they potentiate the activity of L-dopa so that levels of L-dopa with reduced propensity to elicit dyskenesias can be given (Chen, Drug News Perspect. 2003, 16, 597; Morelli et al, Drug Dev. Res. 2001, 52, 387; Bara-Jimenez et al, Neurology 2003, 61, 293). Furthermore, the efficacy of A₂ₐ antagonists does not diminish upon repeated exposure, as seen for L-dopa (Halldner et al, Eur. J. Pharmacol. 2000, 406, 345). A distinct advantage of A₂ₐ antagonists over L-dopa is the propensity for neuroprotection (Morelli et al, Neurotox. Res. 2001, 3, 545).

The adenosine receptor antagonists of the present invention are useful in effecting neuroprotection and in treating central nervous system and peripheral nervous system diseases, neurodegenerative diseases, cardiovascular diseases, cognitive disorders, CNS injury, renal ischemia; acute and chronic pain; affective disorders; cognitive disorders; central nervous system injury; cerebral ischemia; myocardial ischemia; muscle ischemia; sleep disorders; eye disorders and diabetic neuropathy;

In some embodiments the CNS and PNS disorders are movement disorders. A movement disorder may be selected from a disorder of the basal ganglia which results in dyskinesias. Non-limitative disorders include Huntington's disease, multiple system atrophy, progressive supernuclear palsy, essential tremor, myoclonus, corticobasal degeneration, Wilson's disease, progressive pallidal atrophy, Dopa-responsive dystoma-Parkinsonism, spasticity, Alzheimer's disease and Parkinson's disease.

Parkinson's disease further includes early-onset Parkinson's disease, drug-induced Parkinsonism, post-encephalitic Parkinsonism, Parkinsonism induced by poisoning and post-traumatic Parkinson's disease.

The compounds of the present invention have utility as neuroprotectants and may be useful in preventing or treating traumatic brain injury (TBI) and for the attenuation of cognitive impairment in coronary artery bypass graft (CABG) patients. As such the compounds and compositions may be administered to a subject at risk of neural ischemia.

The following examples will further describe the invention, and are used for the purposes of illustration only, and should not be considered as limiting the invention being disclosed.

Abbreviations: The following abbreviations and terms have the indicated meaning throughout, unless otherwise stated:

Ac—acetyl
AcOH—Acetic acid
Boc—tert-butoxycarbonyl
Boc₂O—tert-butoxycarbonic anhydride
Bu—butyl
C—carbon
c—cyclo
CDCl₃—Deuterated chloroform
CD₃OD—Deuterated methanol
δ—NMR chemical shift referenced to tetramethylsilane
DCE—1,2-dichloroethane
DCM—dichloromethane=methylene chloride=CH₂Cl₂
DIC—Diisopropyl carbodiimide

### DIPEA—Diisopropylethylamine

DMAP—4-Dimethylamino pyridine

### DMF—N,N-dimethylformamide

DMSO—Dimethyl sulfoxide
EDC—N-(3-Dimethylaminopropyl)ethylcarbodiimide hydrochloride salt

### Et—Ethyl

EtOAc—Ethyl acetate
ESI—Electrospray ionization

### Et₃N—Triethylamine

### Et₃SiH—Triethylsilane

### ¹H NMR—Proton Nuclear Magnetic Resonance

h—hours
HATU—O-(7-Azabenzotriazol-1-yl)-1,1,3,3-tetramethyluronium hexafluorophosphate
Hexanes—HPLC grade isomeric hexanes
HOBt—hydroxybenzotriazole
i—iso

### LCMS—Liquid Chromatography Mass Spectroscopy

m—meta
Me—methyl
MeOH—methanol=CH₃OH
min—minutes
n—normal
N—nitrogen

### NMR—Nuclear Magnetic Resonance

NaBH₄—sodium borohydride
NaCNBH₃—sodium cyano borohydride
Na(OAc)₃BH—sodium triacetoxy borohydride
o-—ortho
p-—para

### Ph—Phenyl

### Pr—Propyl

r.t.—room temperature
sat.—saturated
s—secondary
t—tertiary
TFA—trifluoro acetic acid

### THF_tetrahydrofuran

### Synthesis of 2-aminobenzimidazoles

Compounds of formula I were synthesized by means of conventional organic synthesis employing solid-phase and solution-phase chemistries, executable by those skilled in the art. The illustration of examples, but not the limitation, of the synthesis of compounds of formula I is detailed in schemes 1-2.

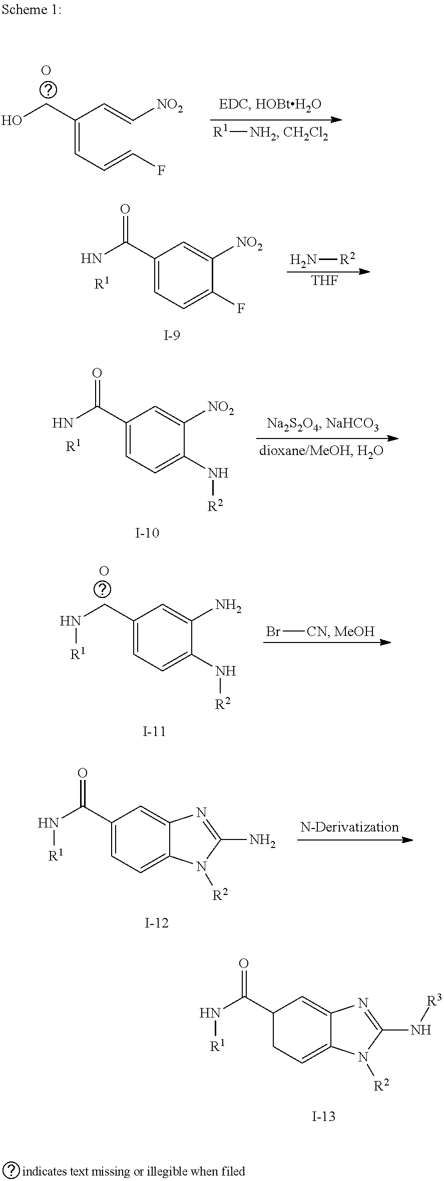

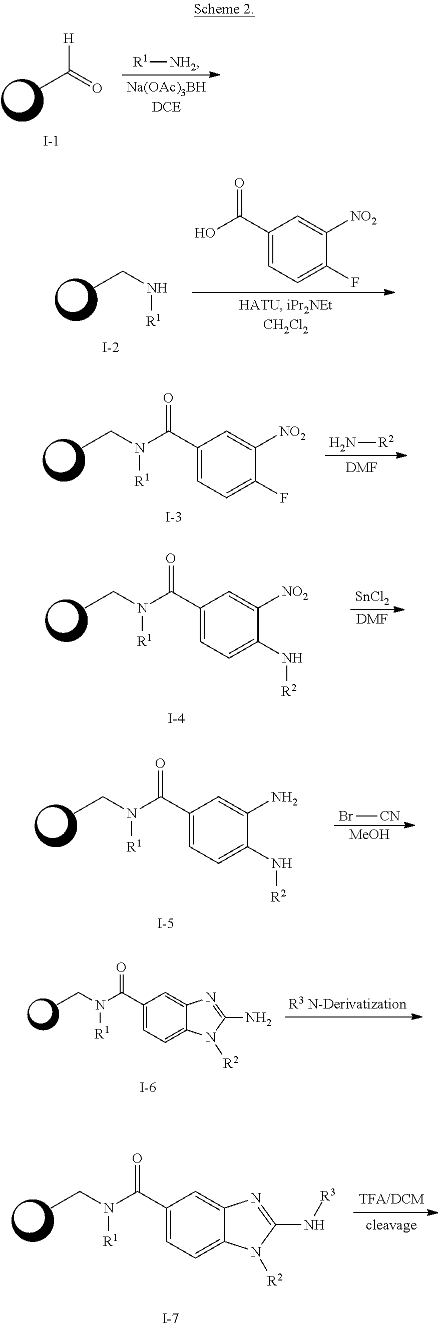

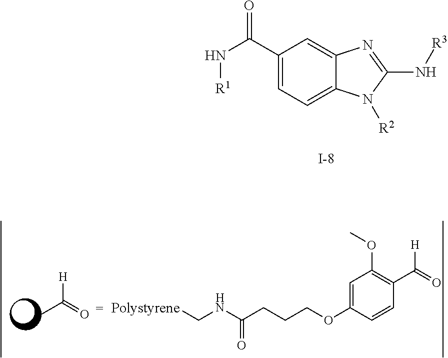

### Solid-Phase Synthesis of Compounds of Formula I

Compounds of formula I (except those in which R¹ is NR⁵R⁷ wherein R⁵ and R⁷, together with the nitrogen atom to which they are attached, are taken together to form a ring) can be synthesized on solid-phase in five steps from 4-(4′-formyl-3′-methoxy)phenoxybutyric acid functionalised amino methyl terminated polystyrene resin utilizing commercially available 4-fluoro-3-nitro benzoic acid (Scheme 2). Reductive alkylation onto the formyl group of the acid labile linker (I-1), followed by amide formation with 4-fluoro-3-nitro benzoic acid provides the carboxamide (I-3). Fluoro displacement with an excess of a primary amine followed by tin chloride mediated nitro reduction and subsequent cyclization with cyanogen bromide provides the aminobenzimidazole (I-6). Functionalization of the 2-amino group with, for example, a carboxylic acid, provides resin-bound compounds of formula I. Ligand cleavage from the solid support is achieved using TFA in CH₂Cl₂, allowing compound purification by flash chromatography or preparative HPLC. Reagents used within the solid-phase synthesis that contain remote nucleophilic functionality can be used in their protected form, for example, but not in limitation, N-Boc protection, O-tBu protection etc. Acid labile protecting groups can be removed during the acid mediated cleavage of the ligand from the solid support.

### Solid-Phase Synthesis—General Procedures

For solid-phase reactions it is often desirable to think of the amount of solution reagents in terms of concentrations rather than equivalents. For this reason, reagent concentration is generally provided in the following solid-phase experimental protocols. All shaking is performed with a wrist-action shaker utilizing a solid-phase synthesis reaction vessel (as described in WO 9711777). The size of shaking vessels typically employed is 20 mL (small) and 100 mL (medium). Each washing cycle is carried out with 12 mL (small vessel) or 60 mL (medium vessel) of solvent over 5-10 minutes unless otherwise stated. All solvents used for reactions and washings are HPLC grade. At most synthetic stages, the resin-bound intermediate can be removed by acid cleavage.

### Intermediate 1 (I-1) General Procedure A: Acylation with 4-(4′-formyl-3′-methoxy)phenoxybutyric acid

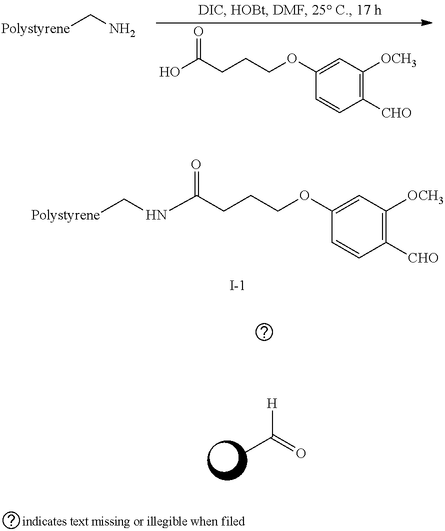

To a solution of 2.86 g (12.0 mmol, 0.2 M, 4.0 eq.) of 4-(4′-formyl-3′-methoxy)phenoxybutyric acid and 1.84 g (12.0 mmol, 0.2 M, 4.0 eq.) of HOBt monohydrate in 60 mL of DMF was added 3.75 mL (24.0 mmol, 0.4 M, 8.0 eq.) of DIC. The resulting solution was stirred for 20 min at 25° C. This solution was added to a medium shaking vessel containing 3.8 g (˜0.8 mmol/g, 3.0 mmol, 1.0 eq.) aminomethyl terminated Polystyrene. The mixture was shaken for 17 h at 25° C. The shaking vessel was then drained and the resin was washed with DMF (1×), CH₂Cl₂ (1×), DMF (2×), CH₂Cl₂ (2×), CH₃OH (2×) and CH₂Cl₂ (2×).

### Intermediate 2 (I-2) General Procedure B: Reductive Amination

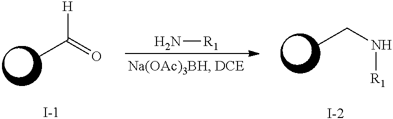

To a suspension of 0.6 g (˜0.8 mmol/g, 0.48 mmol, 1.0 eq.) of resin-bound o-methoxybenzaldehyde (I-1) in 12 mL of 1,2-dichloroethane (DCE) in a small shaking vessel was added 4.8 mmol (0.4 M, 10.0 eq.) of a primary amine. The resin suspension was shaken for 15 sec and 1.0 g (4.8 mmol, 0.4 M, 10.0 eq.) of sodium triacetoxyborohydride was added. The suspension was shaken for 16 h at 25° C., venting the reaction vessel periodically during the first 1 h. The vessel was then drained, and the resin was washed with CH₃OH (1×), CH₂Cl₂ (2×), CH₃OH (1×), CH₂Cl₂ (2×), CH₃OH (1×), CH₃OH (1×30 min) and CH₂Cl₂ (2×).

### Intermediate 3 (I-3) General Procedure C: N-Acylation with 3-nitro-4-fluoro benzoic acid

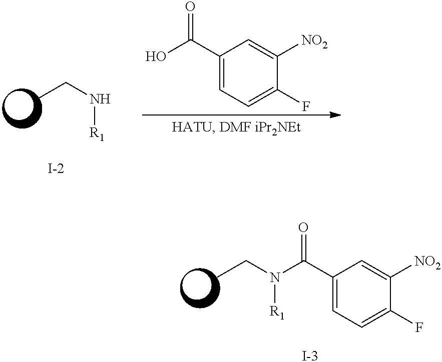

To 0.6 g (˜0.7 mmol/g, 0.4 mmol, 1.0 eq.) of resin-bound secondary amine (I-2) in 10 mL of DMF was added 0.46 g (2.5 mmol, 0.25 M, ˜3.5 eq.) of 3-nitro-4-fluoro benzoic acid and 0.95 g (2.5 mmol, 0.25 M, ˜3.5 eq.) of HATU. A portion of 0.87 mL (5.0 mmol, 0.5 M, ˜7 eq.) of N,N-diisopropylethylamine was added and the mixture was shaken at 25° C. for 16 h. The vessel was drained and the resin was washed with DMF (2×), CH₂Cl₂ (1×), DMF (1×), CH₂Cl₂ (2×), CH₃OH (2×) and CH₂Cl₂ (2×).

### Intermediate 4 (I-4) General Procedure D: N-Arylation with a Primary Amine

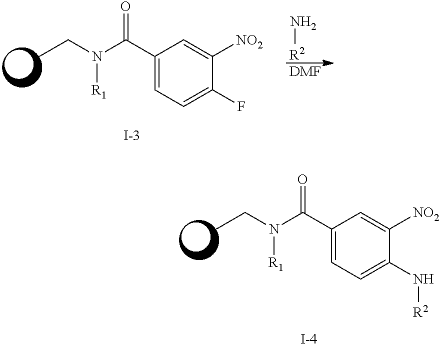

To 0.6 g (˜0.7 mmol/g, 0.4 mmol, 1.0 eq.) of resin-bound aryl fluoride (I-3) in 10 mL of DMF was added 5 mmol (0.5 M, ˜7 eq.) of a primary amine and the mixture was shaken at 25° C. for 16 h. The vessel was drained and the resin was washed with DMF (2×), CH₂Cl₂ (1×), DMF (1×), CH₂Cl₂ (2×), CH₃OH (2×) and CH₂Cl₂ (2×).

### Intermediate 5 (I-5) General Procedure E: Nitro Reduction

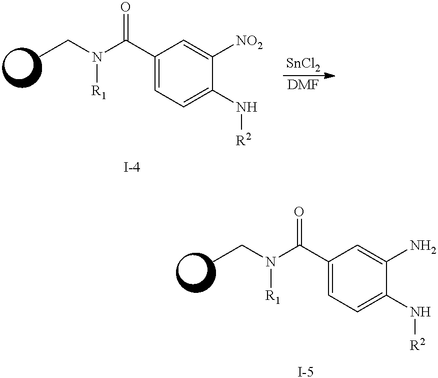

To 0.6 g (˜0.7 mmol/g, 0.4 mmol, 1.0 eq.) of resin-bound nitro compound (I-4) was added 10 mL of a 2 M solution of tin (II) chloride dihydrate in DMF and the mixture was shaken at 25° C. for 36 h. The vessel was drained and the resin was washed with DMF (2×), CH₂Cl2 (1×), DMF (1×), CH₂Cl₂ (2×), CH₃OH (2×) and CH₂Cl₂ (2×).

### Intermediate 6 (I-6) General Procedure F: Aminobenzimidazole Formation

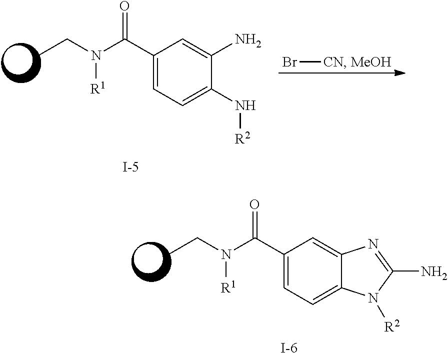

To 0.6 g (˜0.7 mmol/g, 0.4 mmol, 1.0 eq.) of resin-bound aniline (I-5) in 10 mL of MeOH was added 0.53 g (5.0 mmol, 0.5 M, ˜7 eq.) of cyanogen bromide. The mixture was shaken at 25° C. for 16 h. The vessel was drained and the resin was washed with MeOH (2×), DMF (1×), CH₂Cl₂ (2×), CH₃OH (2×) and CH₂Cl₂ (2×).

### Intermediate 7 (I-7) General Procedure G: N-Derivatization

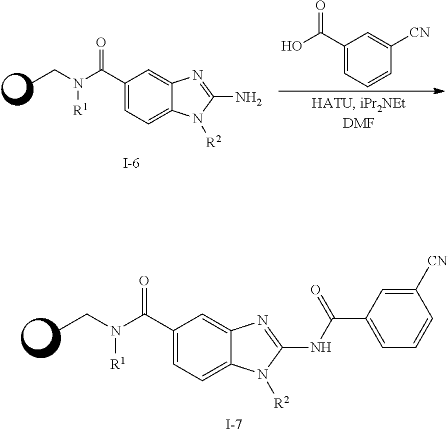

To 0.6 g (˜0.7 mmol/g, 0.4 mmol, 1.0 eq.) of resin-bound aminobenzimidazole (I-6) in 10 mL of DMF was added 0.37 g (2.5 mmol, 0.25 M, ˜3.5 eq.) of 3-cyano benzoic acid and 0.95 g (2.5 mmol, 0.25 M, ˜3.5 eq.) of HATU. A portion of 0.87 mL (5.0 mmol, 0.5 M, ˜7 eq.) of N,N-diisopropylethylamine was added and the mixture was shaken at 25° C. for 16 h. The vessel was drained and the resin was washed with DMF (2×), CH₂Cl₂ (1×), DMF (1×), CH₂Cl₂ (2×), CH₃OH (2×) and CH₂Cl₂ (2×).

### Intermediate 8 (I-8) General Procedure H: Acid Cleavage

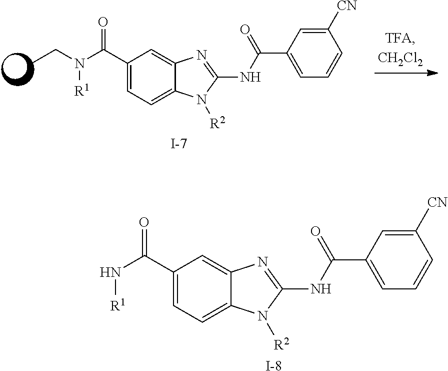

To 0.2 g of resin bound substituted aminobenzimidazole (I-7) in a scintillation vial was added 10 mL of 50% v/v TFA/CH₂Cl₂, and the resulting resin suspension was stirred at room temperature for 2 h. The resin was removed by filtration and the solvent removed in vacuo. The residue was purified by flash chromatography or preparative HPLC.

### Solution-Phase Synthesis

Compounds of type I can be synthesized in five steps from commercially available 4-fluoro-3-nitro benzoic acid (Scheme 1). Activation of the carboxylic acid followed by amide formation with an amine provides the carboxamide (I-9). Fluoro displacement with an excess of a primary amine to provide the substituted 4-amino-3-nitro-benzamide (I-10). Nitro-reduction and subsequent cyclization with cyanogen bromide provides the amino benzimidazole (I-12). Functionalization of the 2-amino group of I-12 with a range of N-derivatizing reagents provides compounds of type I. Analogous compounds of type I can be synthesised using similar experimental procedures.

### Intermediate 9 (I-9)—Procedure I: N-Butyl-4-fluoro-3-nitrobenzamide

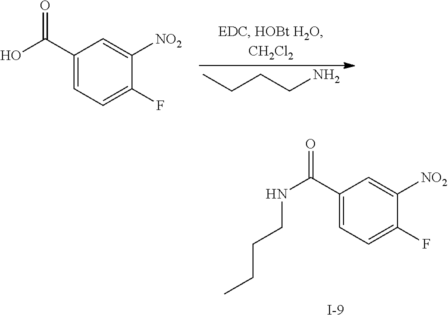

To a solution of 2.0 g (10.8 mmol, 1.0 eq) of 3-nitro-4-fluoro benzoic acid in 30 mL of CH₂Cl₂ was added 2.0 g (12.9 mmol, 1.2 eq.) of HOBt monohydrate and 2.5 g (12.9 mmol, 1.2 eq.) of EDC. A portion of 1.1 mL (10.8 mmol, 1.0 eq.) of n-butyl amine was added and the reaction mixture stirred at room temperature for 30 min. The mixture was diluted with 100 mL of CH₂Cl₂ and washed with 100 mL of sat. NaHCO₃. The organic phase was dried (Na₂SO₄) and the solvent removed in vacuo to provide crude I-9.

### Intermediate 11 (I-11)—Procedure J: 3-Amino-N-butyl-4-(3-methoxypropylamino)benzamide

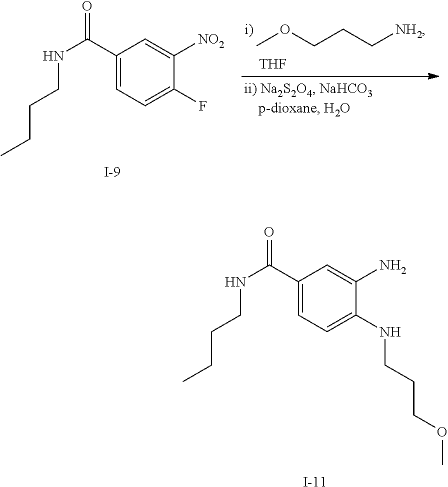

To a solution of 10.8 mmol of crude I-9 in 100 mL of THF was added 8.0 mL of 3-methoxypropyl amine and the mixture stirred at room temperature for 30 min. The solvent was removed in vacuo and the residue re-dissolved in 150 mL of p-dioxane. A solution of 25 g (tech. grade, ˜85%, ˜122 mmol, ˜7 eq.) of sodium hydrosulfite and 10 g (120 mmol, ˜11 eq.) of NaHCO₃ in 200 mL of water was added and the mixture stirred at room temperature for 1 h. The mixture was diluted with 200 mL of EtOAc and 100 mL of sat. NaHCO₃ and the layers separated. The organic phase was dried (Na₂SO₄), and the solvent removed in vacuo to provide 2.7 g of crude I-11.

### Intermediate 12 (I-12)—Procedure K: 2-Amino-N-butyl-1-(3-methoxypropyl)-1H-benzo[d]imidazole-5-carboxamide

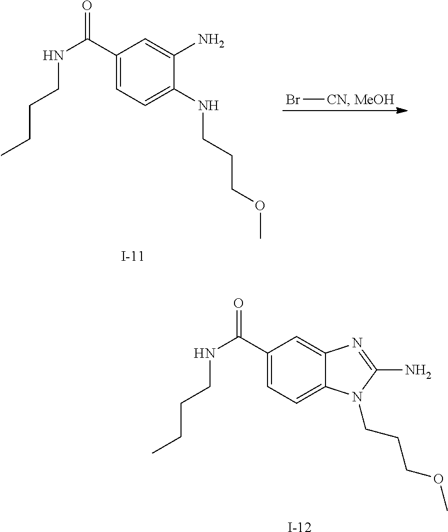

To a solution of 1.5 g of crude I-11 in 20 mL of MeOH was added 0.75 g (7.1 mmol) of cyanogen bromide and the mixture stirred at room temperature for 2 h. The mixture was diluted with 100 mL of EtOAc and washed with 50 mL of sat. Na₂CO₃ and 50 mL of sat. brine. The organic phase was dried (Na₂SO₄) and the solvent was removed in vacuo to provide 1.3 g of crude I-12 as a brown foam. (δ_{H}, 300 MHz, CDCl₃) 0.98 (t, 3H), 1.43 (m, 2H), 1.61 (m, 2H), 2.05 (t, 2H), 3.33 (t, 2H), 3.39 (s, 3H), 3.49 (q, 2H), 4.09 (t, 2H), 6.22 (bt, 1H), 7.10 (d, 1H), 7.58 (dd, 1H), 7.75 (d, 1H); m/z (ESI) found 284.2 [M+H]⁺.

### Intermediate 13 (I-13)—Procedure L: N-Butyl-2-(3-cyanobenzamido)-1-(3-methoxypropyl)-1H-benzo[d]imidazole-5-carboxamide

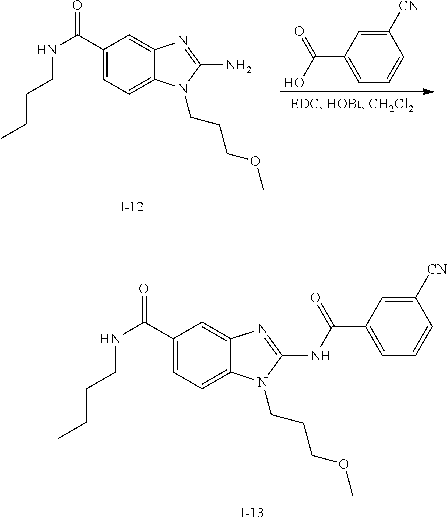

To a solution of 0.44 g of crude I-12 in 10 mL of CH₂Cl₂ was added 0.26 g (1.7 mmol) of 3-cyanobenzoic acid, 0.27 g (1.7 mmol) of HOBt monohydrate and 0.34 g (1.7 mmol) of EDC. The reaction mixture stirred at room temperature for 30 min. The mixture was diluted with 50 mL of CH₂Cl₂ and washed with 50 mL of sat. Na₂CO₃ and 50 mL of 1M HCl. The organic phase was dried (Na₂SO₄) and the solvent removed in vacuo. The residue was purified by flash chromatography (50% EtOAC/hexanes-EtOAc) to provide 128 mg of N-butyl-2-(3-cyanobenzamido)-1-(3-methoxypropyl)-1H-benzo[d]imidazole-5-carboxamide (I-13) as a white solid. δ_{H} (CDCl₃, 300 MHz) 0.98 (t, 3H), 1.42 (m, 2H), 2.20 (m, 2H), 1.65 (m, 2H), 3.33 (s, 3H), 3.41 (t, 2H), 3.50 (dq, 2H), 4.40 (t, 2H), 6.12 (bt, 1H), 7.40 (d, 1H), 7.59 (t, 1H), 7.75 (m, 3H), 8.54 (m, 1H), 8.68 (m, 1H); m/z (ESI) found 434.2 [M+H]⁺.

6-Carboxamido substituted 2-aminobenzimidazoles were synthesized using identical synthetic procedures starting form commercially available 3-fluoro-4-nitro benzoic acid.

7-Carboxamido substituted 2-aminobenzimidazoles were synthesized in six steps starting from commercially available 2-chloro-3-nitro benzoic acid as detailed in Scheme 3.

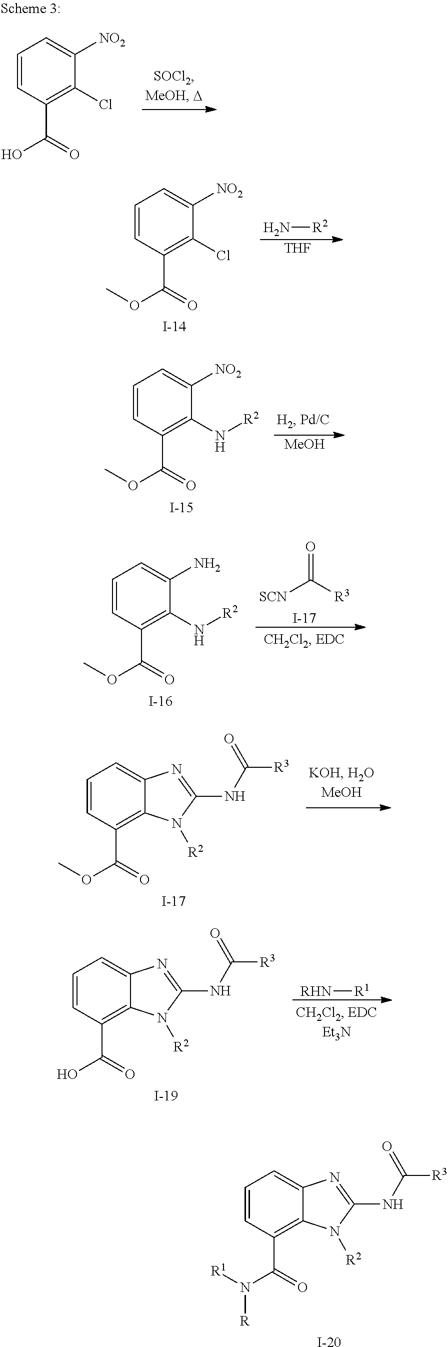

### Intermediate 14 (I-14)—Procedure M: Methyl 2-chloro-3-nitrobenzoate

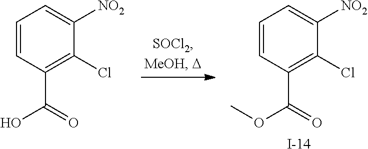

To a solution of 5 g (24 mmol, 1.0 eq.) of 2-chloro-3-nitrobenzoic acid in 50 mL of anhydrous methanol at 0° C. was added 2.74 mL (37 mmol, 1.5 eq.) of thionyl chloride dropwise. The mixture was then heated at reflux for 12 h. The mixture was allowed to cool to room temperature and the solvent removed in vacuo. The residue was redissolved in EtOAc and washed sequentially with 10% NaHCO₃, water and brine. The organic solution was dried (Na₂SO₄) and the solvent removed in vacuo to provide 5.3 g of crude methyl 2-chloro-3-nitrobenzoate (I-14) as a pale yellow solid.

### Intermediate 15 (I-15)—Procedure N: Methyl 2-(3-methoxypropylamino)-3-nitrobenzoate

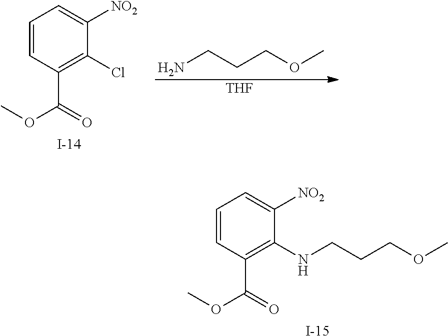

To a solution of 0.5 g (2.3 mmol, 1.0 eq.) of methyl 2-chloro-3-nitrobenzoate (I-14) in 15 mL of anhydrous THF was added 0.38 mL (2.8 mmol, 1.2 eq.) of triethylamine followed by 0.28 mL (2.7 mmol, 1.2 eq.) of 3-methoxypropyl amine. The mixture was then heated at reflux for 12 h. The mixture was allowed to cool to room temperature and the solvent removed in vacuo. The product was isolated by flash chromatography eluting with EtOAc/petroleum ether to provide 0.6 g (2.2 mmol, 96%) of methyl 2-(3-methoxypropylamino)-3-nitrobenzoate (I-15) as a yellow oil.

### Intermediate 16 (I-16)—Procedure O: Methyl 3-amino-2-(3-methoxypropylamino)benzoate

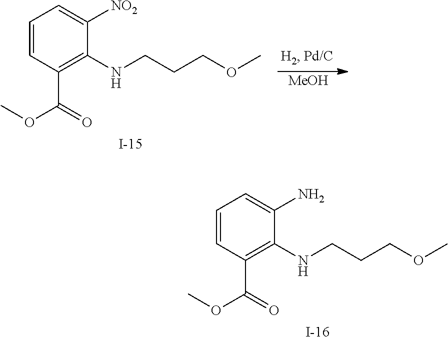

To a solution of 0.6 g (2.2 mmol, 1.0 eq.) of methyl 2-(3-methoxypropylamino)-3-nitrobenzoate (I-15) in 25 mL of MeOH was added 0.06 g of 10% palladium on charcoal. The mixture was stirred under a hydrogen atmosphere for 12 h. The mixture was filtered through Celite® and the pad washed with 5 mL of MeOH. The solvent removed in vacuo to provide 0.5 g of crude methyl 3-amino-2-(3-methoxypropylamino)benzoate (I-16) as a brown liquid oil, which was used without further purification.

### Intermediate 17 (I-17)—Procedure P: 3-Cyanobenzoyl isothiocyanate

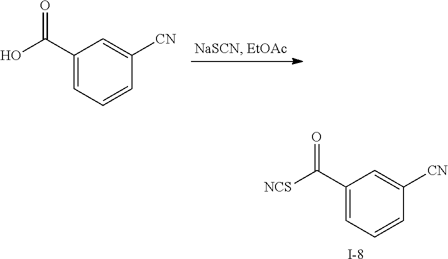

To a solution of 4.0 g (24 mmol. 1.0 eq.) of 3-cyanobenzoyl chloride in 100 mL of EtOAc was added 4.9 g (60 mmol, 2.5 eq.) of sodium thiocyanate and the mixture was stirred at room temperature for 16 h. The suspension was then diluted with 100 mL of hexanes and filtered through Celite®. The filtrate was concentrated in vacuo and the residue purified by flash chromatography (50% EtOAc/hexanes) to provide 3.5 g (18.6 mmol, 78%) of 3-cyanobenzoyl isothiocyanate (I-17) as pale a yellow solid.

### Intermediate 18 (I-18)—Procedure Q: Methyl 2-(3-cyanobenzamido)-3-(3-methoxypropyl)-3H-benzo[d]imidazole-4-carboxylate

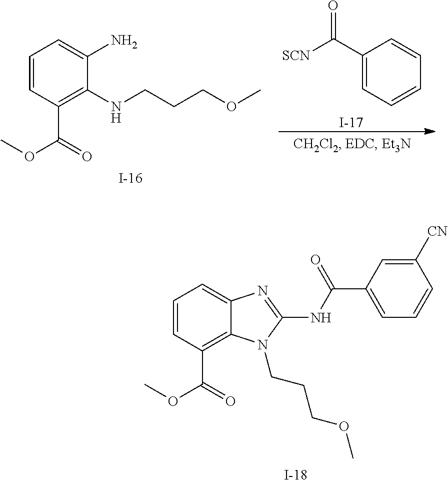

To a solution of 0.25 g (1.0 mmol, 1.0 eq.) of methyl 3-amino-2-(3-methoxypropylamino)benzoate (I-16) in 10 mL of methylene chloride was added 0.22 g (1.1 mmol, 1.1 eq.) of 3-cyanobenzoyl isothiocyanate (I-17) and the mixture was stirred at room temperature for 5 min. A portion of 0.4 g (2.1 mmol, 2.1 eq.) of EDC was added, followed by 0.58 mL (4.2 mmol, 4.2 eq.) of triethylamine and the mixture was stirred at room temperature overnight. The mixture was diluted with 50 mL of methylene chloride and washed sequentially with 10% sodium carbonate solution, water and sat. brine. The organic solution was dried (Na₂SO₄) and the solvent removed in vacuo. The residue was purified by flash chromatography, eluting with 25% EtOAc/petroleum ether to provide 0.2 g (0.5 mmol, 50%) of methyl 2-(3-cyanobenzamido)-3-(3-methoxypropyl)-3H-benzo[d]imidazole-4-carboxylate (I-18) as a white solid.

### Intermediate 19 (I-19)—Procedure R: 2-(3-Cyanobenzamido)-3-(3-methoxypropyl)-3H-benzo[d]imidazole-4-carboxylic acid

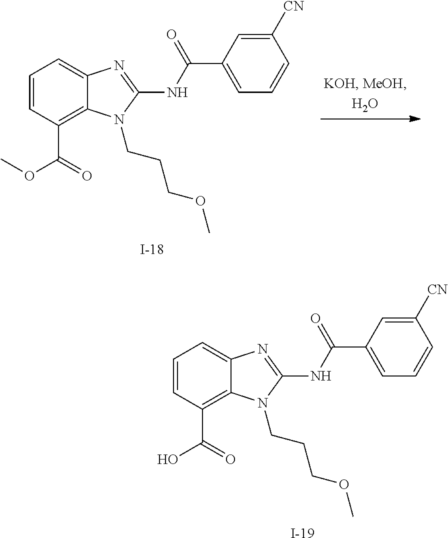

To a solution of 0.2 g (0.5 mmol, 1.0 eq.) of methyl 2-(3-cyanobenzamido)-3-(3-methoxypropyl)-3H-benzo[d]imidazole-4-carboxylate (I-18) in 2 mL of MeOH was added a solution of 0.08 g (1.5 mmol, 3.0 eq.) of potassium hydroxide in 2 mL of water. The mixture was stirred at room temperature for 24 h. The solvent was removed in vacuo and the residue was resuspended in 5 mL of water. The aqueous solution was washed with EtOAc and acidified to pH 4 with 20% citric acid solution. The precipitated white solid was collected, washed with water and dried to provide 0.16 g (0.4 mmol, 83%) of 2-(3-cyanobenzamido)-3-(3-methoxypropyl)-3H-benzo[d]imidazole-4-carboxylic acid (I-19).

### Intermediate 20 (I-20)—General Procedure S

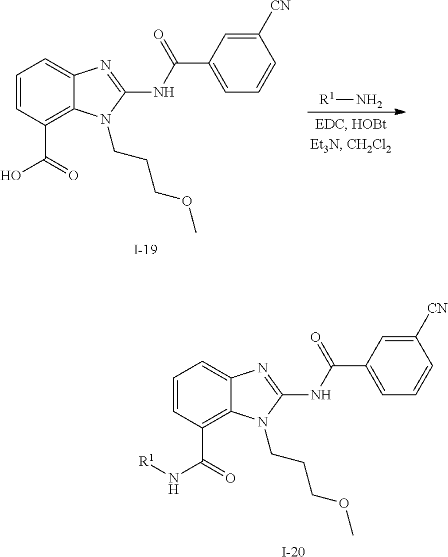

To a solution of 1.0 eq. of I-19 and 1.1 eq. of a primary amine (R¹—NH₂) in methylene chloride was added 1.5 eq. of EDC and 1.5 eq. of HOBt monohydrate, followed by 3.0 eq. of triethylamine. The mixture was then stirred at room temperature for 12 h. The mixture was diluted with methylene chloride and washed sequentially with 1.5 M HCl, 10% NaHCO₃ solution and sat. brine. The organic solution was dried (Na₂SO₄) and the solvent removed in vacuo. The residue was purified by flash chromatography, to provide I-20.

### Analysis—Analytical HPLC Analysis:

Method A: Waters Millenium 2690/996PDA separations system employing a Phenomonex Luna 3u C8 50×4.6 mm analytical column. The aqueous acetonitrile based solvent gradient involves: 0-1 min—Isocratic 10% of (0.1% TFA/acetonitrile); 1 min-7 mm—Linear gradient of 10-90% of (0.1% TFA/acetonitrile) 7 min-9 min—Isocratic 90% of (0.1% TFA/acetonitrile); 9 min-10 min—Linear gradient of 90-10% of (0.1% TFA/acetonitrile); 10 min-12 min—Isocratic 10% of (0.1% TFA/acetonitrile). Flow rate=1 mL/min

Method B: Waters Millenium 2690/996PDA separations system employing a Phenomenex Columbus 5u c18 column 50×4.60 mm analytical column. The aqueous acetonitrile based solvent gradient involves: 0-0.5 min—Isocratic 10% of (0.05% TFA/acetonitrile); 0.5 min-5.5 min—Linear gradient of 10-90% of (0.05% TFA/acetonitrile): 5.5 min-7.5 min—Isocratic 90% of (0.05% TFA/acetonitrile); 7.5 min-8 min—Linear gradient of 90-10% of (0.05% TFA/acetonitrile); 8 min-10 min—Isocratic 10% of (0.05% TFA/acetonitrile). Flow rate=0.4 mL/min

### Analysis—Spectroscopy

Mass Spectroscopy was conducted using a Thermo-electron LCQ classic or an Applied Biosciences PE Sciex API150ex. Liquid Chromatography Mass Spectroscopy was conducted using a Waters Millenium 2690/996PDA linked Thermo-electron LCQ classic. ¹H NMR spectroscopy was conducted using a Varian 300 MHz Gemini 2000 FTNMR. The NMR spectra are tabulated below:

### Methyl 2-(3-cyanobenzamido)-1-(3-methoxypropyl)-1H-benzo[d]imidazole-5-carboxylate

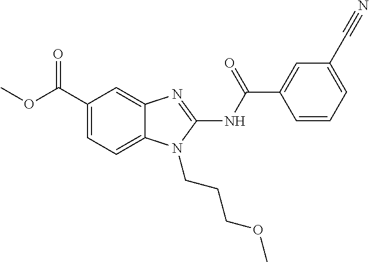

δ_{H} (CDCl₃, 300 MHz) 2.17 (m, 2H), 3.31 (s, 3H), 3.38 (t, 2H), 3.93 (s, 3H), 4.38 (t, 2H), 7.37 (d, 1H), 7.54 (m, 1H), 7.75 (m, 1H), 8.03 (m, 2H), 8.50 (m, 1H), 8.65 (m, 1H), 12.30 (bs, 1H); m/z (ESI) found 393.2 [M+H]⁺

### 2-(3-Cyanobenzamido)-1-(3-methoxypropyl)-N-(2-phenoxyethyl)-1H-benzo[d]imidazole-5-carboxamide

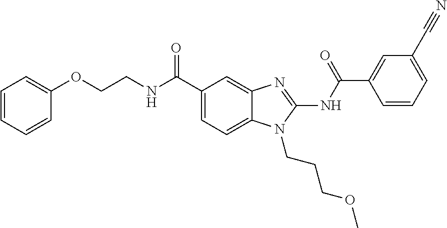

δ_{H} (CDCl₃, 300 MHz) 2.16 (m, 2H), 3.32 (s, 3H), 3.38 (t, 2H), 3.91 (m, 2H), 4.18 (t, 2H), 4.38 (t, 2H), 6.75 (bt, 1H), 6.95 (m, 3H), 7.33 (m, 3H), 7.73 (m, 2H), 7.81 (s, 1H), 8.52 (d, 1H), 8.65 (s, 1H); m/z (ESI) found 498.2 [M+H]⁺

### N-Butyl-2-(3-cyanobenzamido)-1-(3-methoxypropyl)-1H-benzo[d]imidazole-5-carboxamide

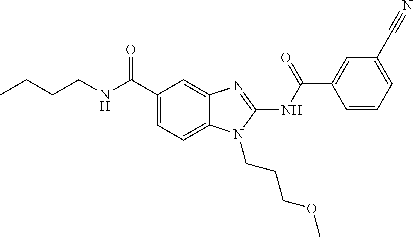

δ_{H} (CDCl₃, 300 MHz) 0.96 (t, 3H), 1.41 (m, 2H), 1.61 (m, 2H), 2.16 (m, 2H), 3.31 (s, 3H), 3.38 (m, 2H), 3.45 (m, 2H), 4.37 (t, 2H), 6.40 (bs, 1H), 7.36 (d, 1H), 7.54 (t, 1H), 7.75 (m, 3H), 8.52 (d, 1H), 8.63 (s, 1H); m/z (ESI) found 434.5 [M+H]⁺.

### N-(3-Methoxybenzyl)-2-(3-cyanobenzamido)-1-(3-methoxypropyl)-1H-benzo[d]imidazole-5-carboxamide

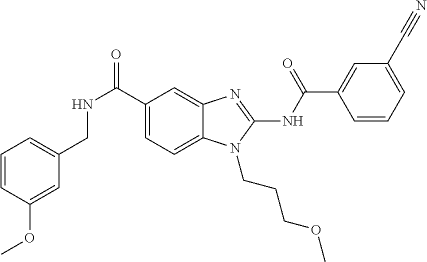

δ_{H} (CDCl₃, 300 MHz) 2.17 (m, 2H), 3.32 (s, 3H), 3.39 (t, 2H), 3.80 (s, 3H), 4.38 (m, 2H), 4.63 (d, 2H), 6.59 (bs, 1H), 6.85 (m, 4H), 7.27 (t, 1H), 7.37 (d, 1H), 7.72 (m, 3H), 8.55 (m, 2H); m/z (ESI) found 498.2 [M+H]⁺.

### 2-(3-Cyanobenzamido)-N-(2-methoxyethyl)-1-(3-methoxypropyl)-1H-benzo[d]imidazole-5-carboxamide

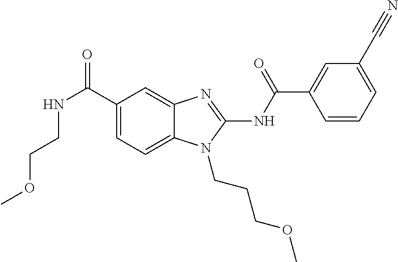

δ_{H} (CDCl₃, 300 MHz) 2.16 (m, 2H), 3.31 (s, 3H), 3.37 (m, 2H), 3.40 (s, 3H), 3.59 (t, 2H), 3.67 (m, 2H), 4.37 (t, 2H), 6.70 (bt, 1H), 7.36 (d, 1H), 7.53 (t, 1H), 7.72 (d, 2H), 7.82 (s, 1H), 8.50 (d, 1H, 8.63 (s, 1H); m/z (ESI) found 436.1 [M+H]⁺.

### N-(2,4-Difluorobenzyl)-2-(3-cyanobenzamido)-1-(3-methoxypropyl)-1H-benzo[d]imidazole-5-carboxamide

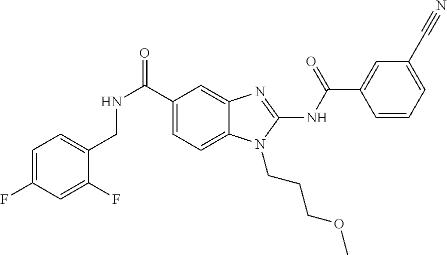

δ_{H} (CDCl₃, 300 MHz) 2.18 (m, 2H), 3.32 (s, 3H), 3.39 (t, 2H), 4.4 (t, 2H), 4.68 (d, 2H), 6.53 (t, 1H), 6.85 (m, 2H), 7.46 (m, 3H), 7.75 (m, 3H), 8.52 (d, 1H), 8.66 (s, 1H); m/z (ESI) found 504.2 [M+H]⁺

### N-(3-Methoxybenzyl)-2-(3-methoxybenzamido)-1-(3-methoxypropyl)-1H-benzo[d]imidazole-5-carboxamide

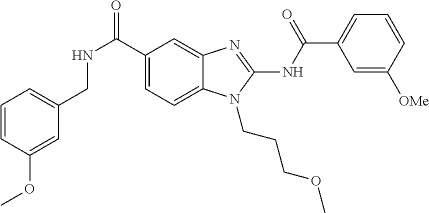

δ_{H} (CDCl₃, 300 MHz) 2.18 (bt, 2H), 3.30 (s, 3H), 3.38 (t, 2H), 3.80 (s, 3H), 3.86 (s, 3H), 4.37 (bt, 2H), 4.59 (s, 2H), 6.43 (bs, 1H), 6.87 (m, 3H), 7.04 (d, 1H), 7.29 (m, 3H), 7.75 (m, 3H), 7.92 (s, 1H), 7.98 (d, 2H); m/z (ESI) found 503.2 [M+H]⁺

### N-(3-Methoxybenzyl)-2-(3-chlorobenzamido)-1-(3-methoxypropyl)-1H-benzo[d]imidazole-5-carboxamide

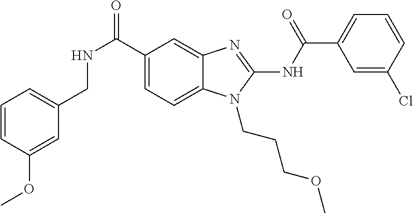

δ_{H} (CDCl₃, 300 MHz) 2.17 (m, 2H), 3.31 (s, 3H), 3.38 (m, 2H), 3.77 (m, 3H), 4.42 (m, 4H), 6.82 (m, 5H), 7.35 (m, 3H), 7.31 (t, 1H), 7.74 (bs, 2H), 8.23 (m, 2H); m/z (ESI) found 507.2 [M+H]⁺

### N-(3-Methoxybenzyl)-2-(3-fluorobenzamido)-1-(3-methoxypropyl)-1H-benzo[d]imidazole-5-carboxamide

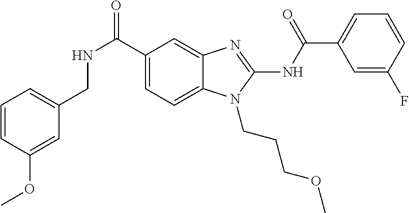

δ_{H} (CDCl₃, 300 MHz) 2.16 (m, 2H), 3.35 (m, 5H), 3.76 (m, 3H), 4.46 (m, 4H), 6.48 (m, 1H), 6.81 (m, 4H), 7.14 (t, 1H), 7.33 (m, 3H), 7.75 (m, 2H), 8.06 (m, 2H); m/z (ESI) found 491.2 [M+H]⁺

### N-(3-Methoxybenzyl)-2-(4-cyanobenzamido)-1-(3-methoxypropyl)-1H-benzo[d]imidazole-5-carboxamide

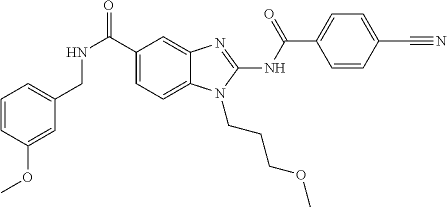

δ_{H} (CDCl₃, 300 MHz) 2.18 (m, 2H), 3.31 (s, 3H), 3.39 (t, 2H), 3.83 (s, 3H), 4.68 (d, 2H), 6.39 (bt, 1H), 6.93 (m, 3H), 7.31 (t, 1H), 7.39 (d, 1H), 7.74 (d, 3H), 7.85 (s, 1H), 8.43 (d, 2H); m/z (ESI) found 498.2 [M+H]⁺

### N-(3-Methoxybenzyl)-2-(3,5-dimethoxybenzamido)-1-(3-methoxypropyl)-1H-benzo[d]imidazole-5-carboxamide

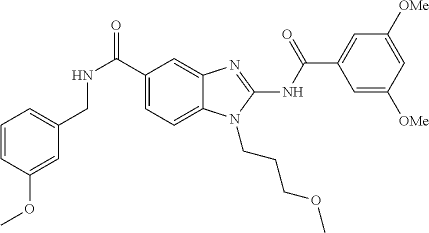

δ_{H} (CDCl₃, 300 MHz) 2.17 (t, 2H), 3.29 (s, 3H), 3.37 (t, 2H), 3.80 (s, 3H), 3.84 (s, 6H), 4.36 (t, 2H), 4.60 (d, 2H), 6.40 (bt, 1H), 6.60 (s, 1H), 6.88 (m, 3H), 7.30 (m, 3H), 7.55 (d, 2H), 7.75 (d, 2H); m/z (ESI) found 533.2 [M+H]⁺

### N-(3-Methoxybenzyl)-2-(2-chlorobenzamido)-1-(3-methoxypropyl)-1H-benzo[d]imidazole-5-carboxamide

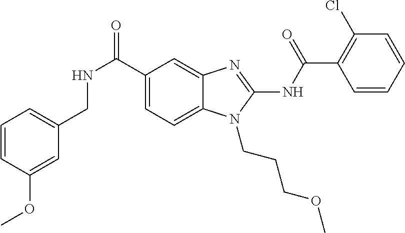

δ_{H} (CDCl₃, 300 MHz) 2.14 (m, 2H), 3.30 (s, 3H), 3.36 (t, 2H), 3.82 (s, 3H), 4.32 (t, 2H), 4.65 (d, 2H), 6.47 (t, 1H), 6.85 (m, 1H), 6.95 (m, 2H), 7.34 (m, 5H), 7.76 (m, 2H), 7.99 (m, 1H); m/z (ESI) found 507.1 [M+H]⁺.

### N-(3-Methoxybenzyl)-2-(furan-2-carboxamido)-1-(3-methoxypropyl)-1H-benzo[d]imidazole-5-carboxamide

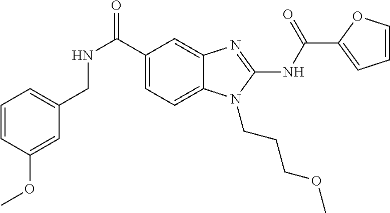

δ_{H} (CDCl₃, 300 MHz) 2.13 (m, 2H), 3.30 (s, 3H), 3.38 (t, 2H), 3.81 (s, 3H), 4.32 (t, 2H), 4.66 (d, 2H), 6.39 (bt, 1H), 6.50 (m, 1H), 6.85 (m, 1H), 6.95 (m, 2H), 7.30 (m, 3H), 7.58 (m, 1H), 7.73 (m, 1H), 7.80 (m, 1H); m/z (ESI) found 463.1 [M+H]⁺

### 3-Cyano-N-(1-(3-Methoxypropyl)-6-(piperidine-4-carbonyl)-1H-benzo[d]imidazol-2-yl)benzamide

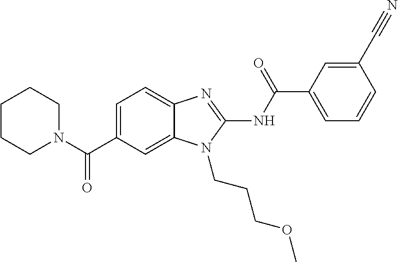

(δ_{H}, 300 MHz, CDCl₃) 1.65 (m, 6H), 2.15 (m, 2H), 3.31 (s, 3H), 3.41 (t, 4H), 3.75 (m, 2H), 4.40 (t, 2H), 7.31 (dd, 1H), 7.43 (d, 1H), 7.51 (s, 1H), 7.56 (t, 1H), 7.77 (d, 1H), 8.48 (d, 1H), 8.62 (s, 1H); m/z (ESI) found 446.2 [M+H]⁺.

### 3-Cyano-N-(1-(3-methoxypropyl)-6-(morpholine-4-carbonyl)-1H-benzo[d]imidazol-2-yl)benzamide

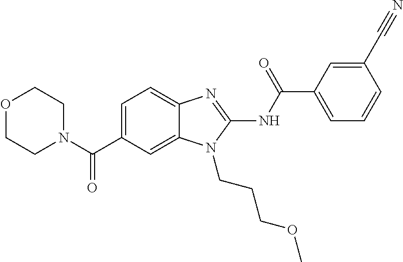

(δ_{H}, 300 MHz, CDCl₃) 2.16 (m, 2H), 3.33 (s, 3H), 3.41 (t, 2H), 3.72 (m, 8H), 4.39 (t, 2H), 7.31 (dd, 1H), 7.39 (d, 1H), 7.52 (m, 1H), 7.56 (t, 1H), 7.77 (dt, 1H), 8.50 (dt, 1H), 8.6 (s, 1H); m/z (ESI) found 448.2 [M+H]⁺.

### N-(6-(Azetidine-1-carbonyl)-1-(3-methoxypropyl)-1H-benzo[d]imidazol-2-yl)-3-cyanobenzamide

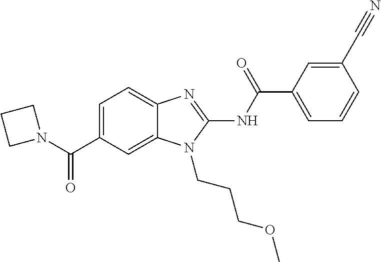

(δ_{H}, 300 MHz, CDCl₃) 2.16 (m, 2H), 2.41 (m, 2H), 3.33 (s, 3H), 3.43 (t, 2H), 4.40 (m, 6H), 7.42 (d, 1H), 7.52 (m, 1H), 7.58 (d, 1H), 7.80 (m, 2H), 8.48 (m, 1H), 8.6 (m, 1H); ESI, found 418.2 [M+H]⁺.

### 2-(3-Cyanobenzamido)-N,N-diethyl-3-(3-methoxypropyl)-3H-benzo[d]imidazol-5-carboxamide

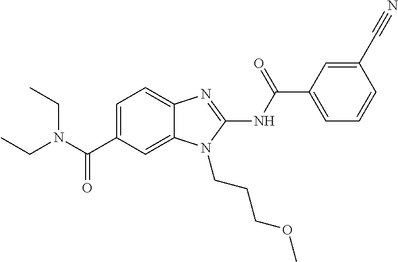

(δ_{H}, 300 MHz, CDCl₃) 1.21 (m, 6H), 2.15 (m, 2H), 3.32 (s, 3H), 3.41 (t, 2H), 3.55 (m, 2H), 4.38 (t, 2H), 7.28 (dd, 1H), 7.38 (d, 1H), 7.45 (m, 1H), 7.55 (dd, 1H), 7.76 (dt, 1H), 8.50 (dt, 1H), 8.64 (m, 1H); ESI, found 434.2 [M+H]⁺.

### Methyl 2-(3-cyanobenzamido)-3-(3-methoxypropyl)-3H-benzo[d]imidazole-4-carboxylate

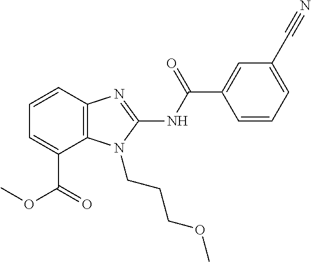

(δ_{H}, 300 MHz, CDCl₃) 2.06 (m, 2H), 3.39 (s, 3H), 3.42 (t, 2H), 4.03 (s, 3H), 4.78 (t, 2H), 7.31 (m, 1H), 7.52 (d, 1H), 7.60 (m, 1H), 7.73 (d, 1H), 7.79 (d, 1H), 8.56 (m, 1H), 8.71 (s, 1H), 12.59 (bs, 1H); ESI, 393.2 [M+H].

### 2-(3-Cyanobenzamido)-3-(3-methoxypropyl)-3H-benzo[d]imidazole-4-carboxylic acid

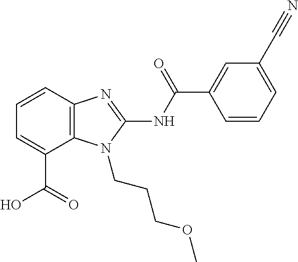

(δ_{H}, 300 MHz, d₆-DMSO) 1.94 (m, 2H), 3.21 (s, 3H), 3.33 (m, 2H), 4.67 (t, 2H), 7.30 (m, 1H), 7.61 (d, 1H), 7.75 (m, 2H), 8.01 (d, 1H), 8.53 (d, 1H), 8.61 (s, 1H), 13.08 (bs 1H); ESI, 379.2 [M+H].

### 3-Cyano-N-(1-(3-methoxypropyl)-7-(morpholine-4-carbonyl)-1H-benzo[d]imidazol-2-yl)benzamide

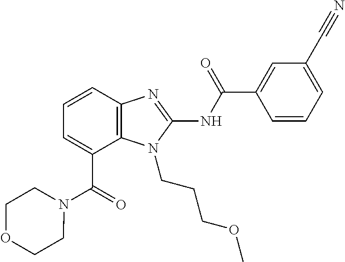

(δ_{H}, 300 MHz, d₆-DMSO) 1.96 (m, 2H), 3.25 (s, 3H), 3.30-3.77 (m, 10H), 4.32 (m, 2H), 7.19 (d, 1H), 7.30 (m, 1H), 7.65 (d, 1H), 7.72 (m, 1H), 8.01 (d, 1H), 8.52 (d, 1H), 8.60 (s, 1H), 13.0 (bs 1H); ESI, 448.2 [M+H].

### 3-Cyano-N-(1-(3-methoxypropyl)-7-(piperidine-1-carbonyl)-1H-benzo[d]imidazol-2-yl)benzamide

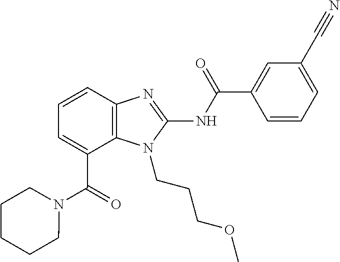

(δ_{H}, 300 MHz, d₆-DMSO) 1.42 (m, 2H), 1.60 (m, 4H), 1.97 (m, 2H), 3.22 (s, 3H), 3.17-3.50 (m, 2H), 3.70 (m, 2H), 4.30 (m, 2H), 7.14 (d, 1H), 7.29 (m, 1H), 7.63 (d, 1H), 7.72 (m, 2H), 8.01 (d, 1H), 8.52 (d, 1H), 8.60 (s, 1H), 13.0 (bs 1H); ESI, 446.2 [M+H].

### 2-(3-Cyanobenzamido)-3-(3-methoxypropyl)-3H-benzo[d]imidazole-4-carboxamide

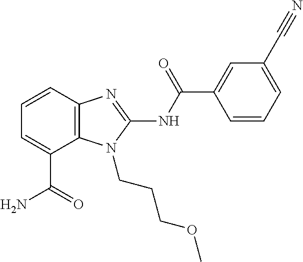

(δ_{H}, 300 MHz, d₆-DMSO) 1.90 (m, 2H), 3.19 (s, 3H), 3.30 (m, 2H), 4.62 (m, 2H), 7.16 (m, 1H), 7.22 (m, 1H), 7.63 (m, 2H), 7.78 (m, 1H), 8.01 (m, 2H), 8.51 (d, 1H), 8.61 (s, 1H), 9.01 (bs 1H); ESI, 378.2 [M+H].

### A₂ₐ Binding Assay:

Membranes prepared from HEK-293 cells that express human A₂ₐ (0.04 mg/mL final, PerkinElmer Life and Analytical Sciences, Boston, Mass.) were mixed with yttrium oxide wheatgerm-agglutinin (WGA)-coated SPA beads (4 mg/mL final, Amersham Biosciences, Piscataway, N.J.) and adenosine deaminase (0.01 mg/mL final) in assay buffer (Dulbecco's phosphate-buffered saline containing 10 mM MgCl₂) for 15 minutes at 4° C. This mixture (10 μL) was added with continuous agitation to the test compounds (10 μL) prepared in 2.5% DMSO or to 2.5% DMSO (1% final) in 384-well assay plates (Corning #3710).

Binding was initiated with the addition of 5 μL of [³H]SCH 58261 (2 nM final, Amersham Biosciences) immediately followed by centrifugation at 1000 rpm for 2 min. The assay plates were incubated in the dark, overnight at room temperature and the signal was detected using a ViewLux CCD Imager (PerkinElmer). Compounds were tested at 11 different concentrations ranging from 0.1 nM to 10 μM. Nonspecific binding was determined in the presence of 10 μM CGS 15943. Assays were performed in duplicate and compounds were tested at least twice. The data were fit to a one-site competition binding model for IC₅₀ determination using the program GraphPad Prism (GraphPad Software, Inc., San Diego, Calif.) and Kᵢ values were calculated using the Cheng-Prusoff equation (Cheng, Y, Prusoff, W. H. Biochem. Pharmacol. 1973, 22, 3099).

### A₁ Binding Assay:

As described in Matasi et al. (Bioorg. Med. Chem. Lett. 2005, 15, 1333), membranes (10 μg) prepared from CHO (Chinese Hamster Ovary) cells that express human A₁ were mixed with 1 nM (final) [³H]DPCPX in 200 μL assay buffer (2.7 mM KCl, 1.1 mM KH₂PO₄, 137 mM NaCl, 7.6 mM Na₂HPO₄, 10 mM MgCl₂, 0.04% methyl cellulose, 20 μg/mL adenosine deaminase) containing 4% DMSO with or without test compounds. Reactions were carried out for 60 min at room temperature and were terminated by rapid filtration over GF/B filters. Filters were washed seven times with 1 mL cold distilled H₂O, air dried, and radioactivity retained on filters were counted in a Packard TopCount® NXT microplate scintillation counter (Global Medical Instrumentation, Inc., Ramsey, Minn.). Compounds were tested at 10 different concentrations ranging from 0.1 nM to 10 μM. Nonspecific binding was determined in the presence of 10 μM NECA (5′-(N-Ethylcarboxamido)adenosine). Assays were preformed in duplicate and compounds were tested two times. Data were fit to a one-site competition binding model for IC₅₀ determination using the program GraphPad Prism (GraphPad Software, Inc., San Diego, Calif.) and Kᵢ values were calculated using the Cheng-Prusoff equation (Cheng, Y, Prusoff, W. H. Biochem. Pharmacol. 1973, 22, 3099).

The results of testing of representative species are shown below. The compounds in Table 1 below exhibited Kᵢ for the A₂ₐ receptor below 10 μM.

TABLE 1

| Example | Compound |
|---|---|
| 1 | 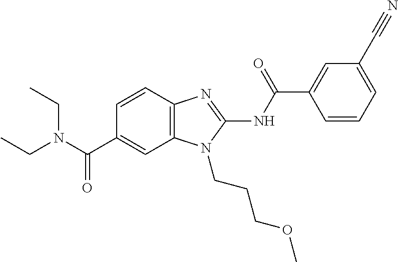 |
| 2 | 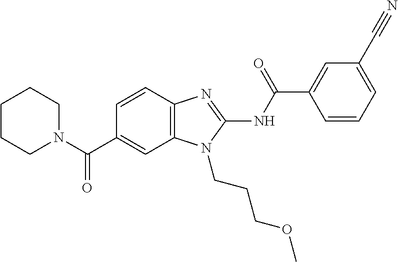 |
| 3 | 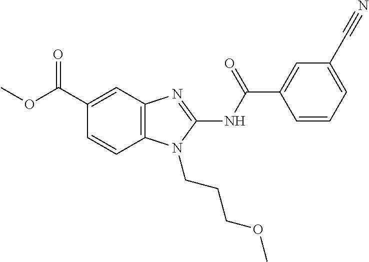 |
| 4 | 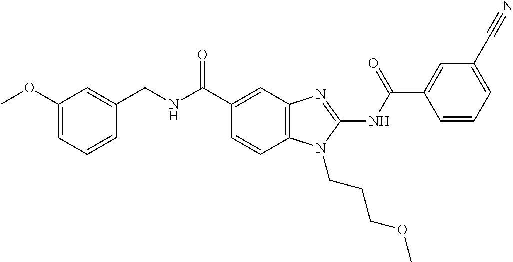 |
| 5 | 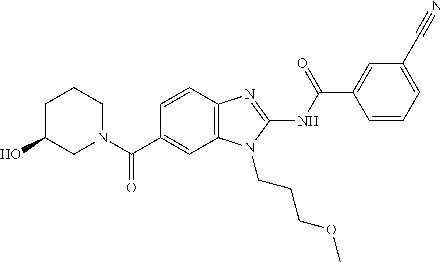 |
| 6 | 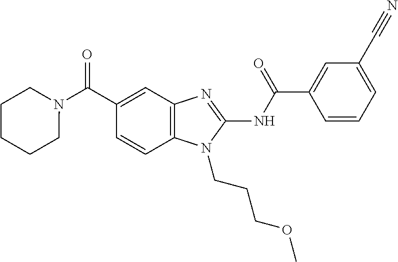 |
| 7 | 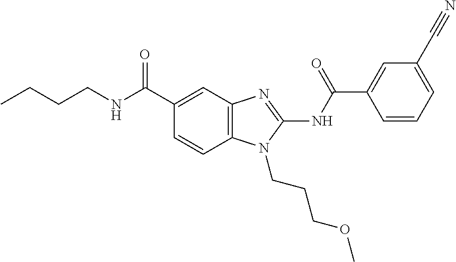 |
| 8 | 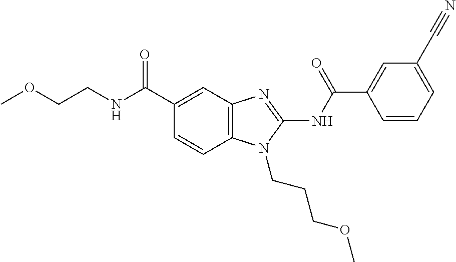 |
| 9 | 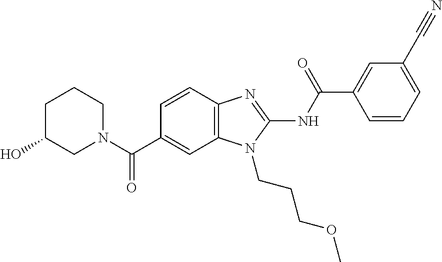 |
| 10 | 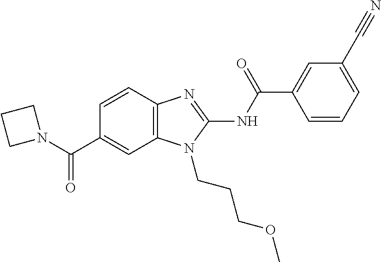 |
| 11 | 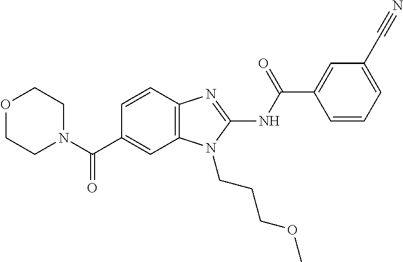 |
| 12 | 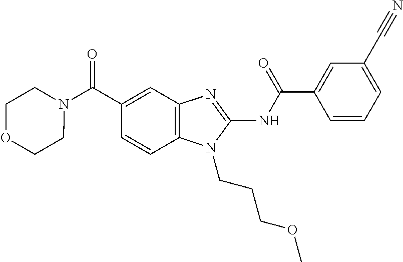 |
| 13 | 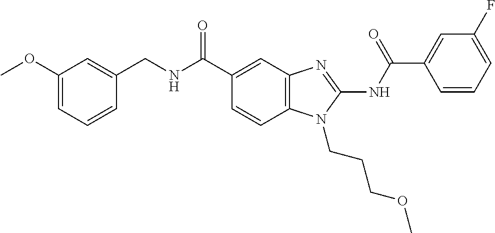 |
| 14 | 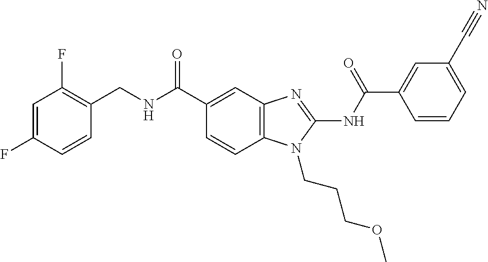 |
| 15 | 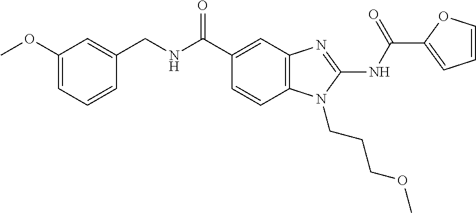 |
| 16 | 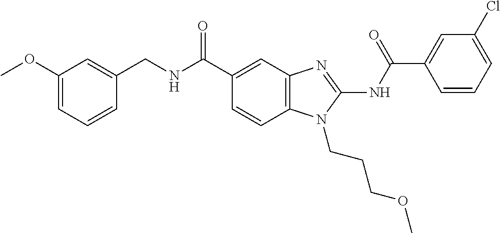 |
| 17 | 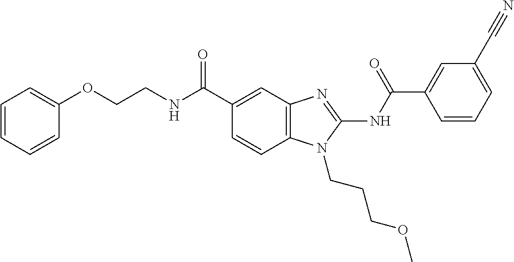 |
| 18 | 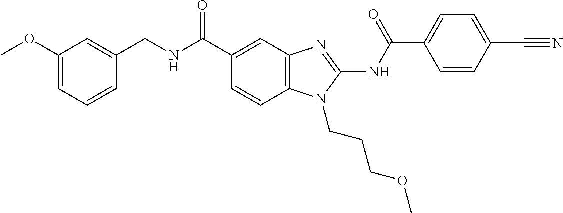 |
| 19 | 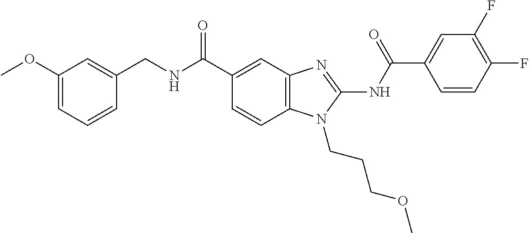 |
| 20 | 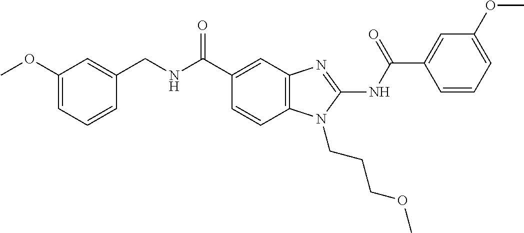 |
| 21 | 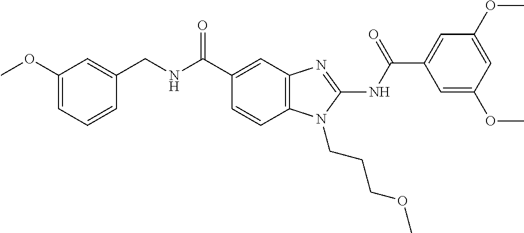 |
| 22 | 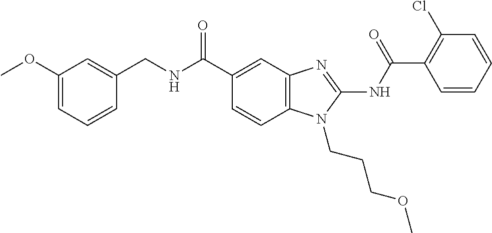 |
| 23 | 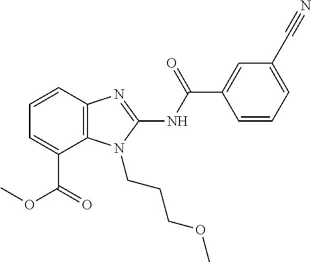 |
| 24 | 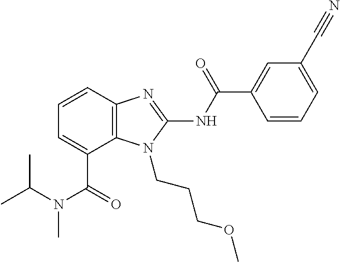 |
| 25 | 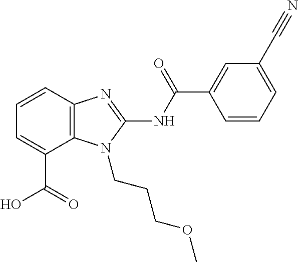 |
| 26 | 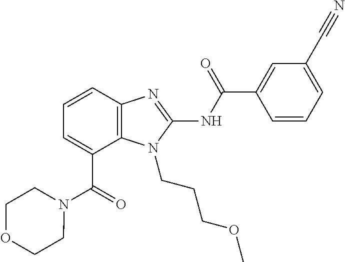 |
| 27 | 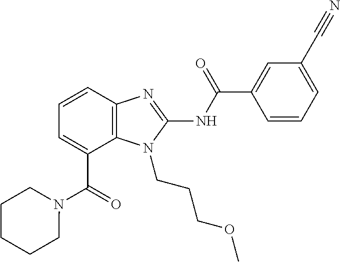 |
| 28 | 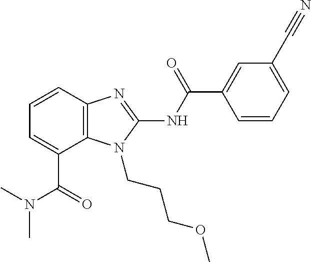 |
| 29 | 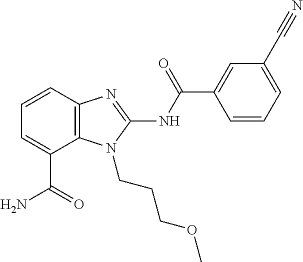 |

Although the foregoing invention has been described in some detail for purposes of illustration, it will be readily apparent to one skilled in the art that changes and modifications may be made without departing from the scope of the invention described herein.

1. A compound of formula I 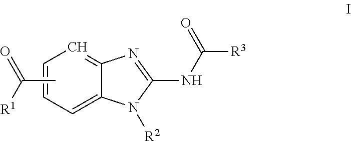
wherein
R¹ is selected from the group consisting of OR⁴, N(R⁵)(CH₂)ₙR⁶ and N(R⁵)R⁷;
R² is selected from the group consisting of C₃-C₂₀ hydrocarbon in which from one to three —CH₂— are replaced by —O—;
R³ is selected from the group consisting of aryl, arylalkyl, heteroaryl, heteroarylalkyl, R⁹-substituted aryl, R⁹-substituted arylalkyl, R⁹-substituted heteroaryl and R⁹-substituted heteroarylalkyl, wherein R⁹ represents from 1 to 3 substituents independently selected from cyano, methyl, methoxy, hydroxy, nitro and halogen;
R⁴ is selected from the group consisting of H, C₁-C₂₀ hydrocarbon, heteroaryl, heteroarylalkyl, substituted alkyl, substituted aryl, substituted arylalkyl, substituted heteroaryl and substituted heteroarylalkyl;
R⁵ is selected from the group consisting of H and C₁-C₄ alkyl;
n is an integer selected from 1-4;
R⁶ is selected from the group consisting of aryl, heteroaryl, substituted aryl and substituted heteroaryl; or
when n is 2, 3 or 4, R⁶ may additionally be alkoxy, aryloxy or substituted aryloxy; and
R⁷ is H or C₁-C₂₀ hydrocarbon,
or R⁵ and R⁷, together with the nitrogen atom to which they are attached, form a 4-7 membered optionally substituted monocyclic ring or an 8-14 membered optionally substituted bicyclic ring, wherein each monocyclic or bicyclic ring optionally contains an additional 1 to 3 heteroatoms chosen from N, O and S;
with the provisos that (1) when R² is —(CH₂)₃—OCH₃, —CO—R¹ is at the 5-position of benzimidazole ring, and R¹ is —N(CH₃)-cyclohexyl, R³ is not thien-2-yl, (2) when R² is —(CH₂)₃—OCH₃, —CO—R¹ is at the 7-position of benzimidazole ring, and R¹ is —NH-benzyl, R³ is not 3-cyanophenyl, and (3) when R² is —(CH₂)₃—OCH₃, —CO—R¹ is at the 5-position of benzimidazole ring, and R¹ is —NH—CH₂-(3-methoxyphenyl), R³ is not 1-cyanocyclopropyl, 3-N,N-dimethylaminophenyl, 3-trifluoromethylphenyl, or 2-methoxyethyl.

2. A compound according to claim 1 of formula Id

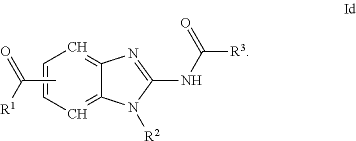

3. A compound according to claim 1 of formula Ie

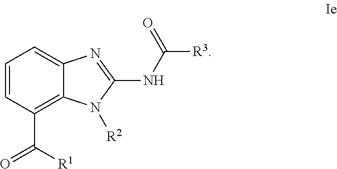

4. A compound according to claim 1 wherein R¹ is OR⁴.

5. A compound according to claim 4 wherein R² is C₃-C₂₀ oxaalkyl and R³ is heteroaryl or substituted phenyl.

6. A compound according to claim 1 wherein R¹ is N(R⁵)(CH₂)ₙR⁶.

7. A compound according to claim 6 wherein R⁵ is H.

8. A compound according to claim 7 wherein n is 1 and R⁶ is substituted aryl.

9. A compound according to claim 7 wherein n is 2 or 3 and R⁶ selected from the group consisting of aryl, heteroaryl, substituted aryl, substituted heteroaryl, alkoxy, aryloxy and substituted aryloxy.

10. A compound according to claim 6 wherein R² is C₃-C₂₀ oxaalkyl and R³ is heteroaryl or substituted phenyl.

11. A compound according to claim 10 wherein R³ is meta-substituted phenyl and R⁹ is cyano.

12. A compound according to claim 1 wherein R¹ is N(R⁵)R⁷.

13. A compound according to claim 12 wherein R⁵ and R⁷ are both C₁-C₆alkyl.

14. A compound according to claim 12 wherein R⁵ and R⁷ are taken together with the nitrogen atom to which they are attached to form a C₄-C₆ optionally substituted monocyclic ring.

15. A compound according to claim 14 wherein said ring is selected from an optionally substituted azetidine ring, an optionally substituted piperidine ring, and an optionally substituted morpholine ring.

16. A compound according to claim 13 wherein R² is C₃-C₂₀ oxaalkyl and R³ is heteroaryl or substituted phenyl.

17. A compound according to claim 16 wherein R³ is meta-substituted phenyl and R⁹ is cyano.

18. A compound according to claim 1 of formula II 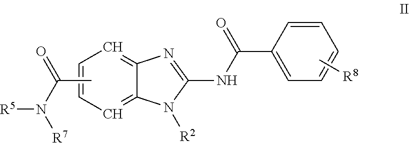
wherein
R⁸ is chosen from halogen, cyano, methoxy, hydroxyl, methyl and nitro.

19. A compound according to claim 1 which is in the form of a pharmaceutically acceptable salt.

20. A pharmaceutical composition comprising a pharmaceutically acceptable carrier and a therapeutically effective amount of at least one compound according to claim 1.

21. A composition according to claim 20 further comprising a second active ingredient selected from the group consisting of: (1) an agent useful in the treatment of Parkinson's disease, (2) an agent useful in the treatment of movement disorders, and (3) an agent useful in the treatment of depression.

22. A composition according to claim 21 wherein said second active ingredient is a dopaminergic receptor agonist.

23. A method of treating a disorder which is mediated by adenosine receptor function, which comprises administering to a subject in need of such treatment a therapeutically effective amount of a compound according to claim 1.

24. A method according to claim 23 wherein the disorder is a disorder associated with adenosine A₂ₐ receptors.

25. A method according to claim 23 wherein the disorder is selected from the group consisting of central nervous system and peripheral nervous system diseases; neurodegenerative diseases; cardiovascular diseases; cognitive disorders; CNS injury; renal ischemia; acute and chronic pain; affective disorders; cognitive disorders; central nervous system injury; cerebral ischemia; myocardial ischemia; muscle ischemia; sleep disorders; eye disorders and diabetic neuropathy.

26. A method according to claim 25 wherein the CNS and PNS disorders are movement disorders.

27. A method according to claim 26 wherein the movement disorder is selected from the group consisting of (1) diskinetic disorders of the basal ganglia; (2) Huntington's disease, (3) multiple system atrophy, (4) progressive supernuclear palsy, (5) essential tremor, (6) myoclonus, (7) corticobasal degeneration, (8) Wilson's disease, (9) progressive pallidal atrophy, (10) Dopa-responsive dystoma-Parkinsonism, (11) spasticity, (12) Alzheimer's disease and (13) Parkinson's disease.

28. A method according to claim 27 wherein the movement disorder is Parkinson's disease.

29. A method according to claim 23 wherein said method is for neuroprotection in a subject at risk of neural ischemia.

30. A method according to claim 23 wherein said method is for treating of injuries to the central nervous system.

31. (canceled)

32. (canceled)
